(19) United States
(12) Patent Application Publication
(10) Pub. No.: US 2022/0110984 A1
(43) Pub. Date: Apr. 14, 2022

(54) Application of Dubosiella Newyorkensis in Prolonging Lifespan and Anti-Aging

(71) Applicant: Jiangnan University, Wuxi (CN)

(72) Inventors: Shenglong Zhu, Wuxi (CN); Yongquan Chen, Wuxi (CN); Xuan Jiang, Wuxi (CN); Zhen Wang, Wuxi (CN)

(21) Appl. No.: 17/559,006

(22) Filed: Dec. 22, 2021

Related U.S. Application Data
(63) Continuation of application No. PCT/CN2020/139830, filed on Dec. 28, 2020.

(30) Foreign Application Priority Data
Jul. 24, 2020 (CN) 2020107229500

(51) Int. Cl. A61K 35/741; A23L 33/135; A23L 33/00; A23K 10/18

(52) U.S. Cl. CPC A61K 35/741; A23L 33/135; A23V 2002/00; A23K 10/18; A23L 33/40

(57) ABSTRACT

The present disclosure discloses application of D. newyorkensis in prolonging lifespan and anti-aging, and belongs to the technical fields of biomedicine and microorganisms. The D. newyorkensis NYU-BL-A4 used in the present disclosure can prolong the lifespan of C. elegans without affecting the normal vital sign of swallowing frequency of C. elegans, is safe and has no toxic and side effects to the nematodes. Therefore, it can have a good anti-aging effect and can be applied to the preparation of anti-aging and life-prolonging products.

### TECHNICAL FIELD

The present disclosure relates to application of Dubosiella newyorkensis in prolonging lifespan and anti-aging, and belongs to the technical fields of biomedicine and microorganisms.

### BACKGROUND

Aging is a biological process shared by all living organisms and triggered by multiple factors, and is characterized by the gradual damage of the response of the host to stress and the general cellular degradation in key metabolic pathways. Aging and many related harmful physiological effects affect the health and overall function of organisms. In humans and other mammals, aging can cause a decline in immune function, increased susceptibility to diseases (cancer, diabetes, cardiovascular disease, and neurodegenerative diseases), increased incidence of chronic inflammation, decreased muscle mass, and increased incidence of Alzheimer's disease, Huntington's disease and other age-related degenerative diseases.

Recent studies have shown that changes in the intestinal microbiota exhibit age-related phenotypes and are related to the aging process of the host. Among intestinal bacteria, probiotics are functional organs in the human body because they have a wide range of positive effects on the health of the host. The effects include regulation of the intestinal microbiota, immune regulation, anti-cancer effects and positive effects on the lifespan of the host. The anti-aging effects of probiotics have been realized as early as 1908, at that time the researchers reported that farmers who consumed large amounts of fermented milk and lactobacilli in Bulgaria had prolonged lifespans.

### SUMMARY

As a new bacterial genus isolated and identified from biological samples, Dubosiella exhibits the effects of regulating metabolism in vivo, improving intestinal immunity, promoting resistance of the body to inflammatory diseases, and the like. It can affect various life activities of individuals.

The lifespan research model animal Caenorhabditis elegans N2 used in the present disclosure is a common small soil nematode. It has short life cycle, fast reproduction time, and has great homology with human biochemical and gene pathways, and its genetic and pharmacological mechanisms for regulating lifespan have been clear. Research on the mechanism of aging and anti-aging, in fact, several major theories of aging mechanism have been proved in nematodes. Therefore, drugs that have anti-aging effects on nematodes are generally considered to have the same effects on humans, and nematodes are therefore used as a common model organism for studying aging.

In order to solve the existing problems, the present disclosure first provides a strain of D. newyorkensis NYU-BL-A4, and the D. newyorkensis NYU-BL-A4 was preserved in Leibniz Institute DSMZ-German Collection of Microorganisms and Cell Cultures, Inhoffenstraße 7B 38124 Braunschweig GERMANY on Jul. 8, 2016 and the preservation number is No. DSM 103457.

The present disclosure discloses application of D. newyorkensis NYU-BL-A4 in preparation of products for prolonging lifespan and/or anti-aging in animals.

In one implementation, the product includes, but is not limited to, food, health-care products, feeds or additives thereof.

In one implementation, the food, health-care products, feeds or additives thereof are used to improve the immunity and strengthen the basal metabolic capacity in the body.

In one implementation, dosage forms of the food, health-care products, feeds or additives thereof include tablets, granules, capsules, powders, liquids, jellies, and sticks.

In one implementation, the food, health-care products, feeds or additives thereof also contain common ingredients in the art, which are appropriately selected by those skilled in the art according to the dosage form or purpose of use, and can be used together with other raw materials.

In one implementation, prolonging lifespan is to prolong the maximum length of the lifespan of the body or delay the time to death.

In one implementation, the animal generally refers to organisms that belong to the animal kingdom in biological classification, and includes, but is not limited to, mammals.

In one implementation, the animal is a nematode.

In one implementation, the content of D. newyorkensis NYU-BL-A4 in the product is not less than 1.5×10⁸ CFU/mL or 1.5×10⁸ CFU/mg.

The present disclosure discloses application of D. newyorkensis in preparation of products for improving the muscle capacity of animals, and the D. newyorkensis is NYU-BL-A4.

In one implementation, the D. newyorkensis NYU-BL-A4 was preserved in Leibniz Institute DSMZ-German Collection of Microorganisms and Cell Cultures, Inhoffenstraße 7B 38124 Braunschweig GERMANY on Jul. 8, 2016 and the preservation number is No. DSM 103457.

In one implementation, the product includes, but is not limited to, food, health-care products, medicines and/or pharmaceutical compositions, feeds or additives thereof.

In one implementation, forms of the food, health-care products, medicines and/or pharmaceutical compositions, feeds or additives thereof include tablets, granules, capsules, powders, liquids, jellies, and sticks.

In one implementation, the food, health-care products, feeds or additives thereof also contain common ingredients in the art, which are appropriately selected by those skilled in the art according to the dosage form or purpose of use, and can be used together with other raw materials.

### Beneficial Effects

The D. newyorkensis NYU-BL-A4 according to the present disclosure can prolong the lifespan of C. elegans without affecting the normal vital sign of swallowing frequency of C. elegans, is safe and has no toxic and side effects to the nematodes, which proves that the D. newyorkensis strain NYU-BL-A4 has a good anti-aging effect and can be used to assist the preparation of anti-aging products. Therefore, it can be applied to alleviate aging, prolong lifespan, has good practical application value, and is suitable for commercial production.

### BRIEF DESCRIPTION OF FIGURES

FIG. 1 is a diagram showing the relationship between D. newyorkensis NYU-BL-A4 and the lifespan of C. elegans.

FIG. 2 is a diagram showing the relationship between D. newyorkensis NYU-BL-A4 and the vital sign of swallowing frequency of C. elegans.

FIG. 3 is a diagram showing the relationship between D. newyorkensis NYU-BL-A4 and safety and toxicity in C. elegans.

FIG. 4 is a diagram showing the relationship between D. newyorkensis NYU-BL-A4 and the total number of eggs laid by C. elegans.

FIG. 5 is a diagram showing the relationship between D. newyorkensis NYU-BL-A4 and the daily number of eggs laid by C. elegans.

FIG. 6 is a diagram showing the relationship between D. newyorkensis NYU-BL-A4 and the dynamic swing frequency of C. elegans.

### DETAILED DESCRIPTION

NGM medium (per liter): peptone 2.5 g, sodium chloride 3.0 g, calcium chloride 0.111 g, magnesium sulfate 0.12 g, cholesterol 0.005 g, potassium dihydrogen phosphate 3.4 g, and agar 17.0 g.

### Example 1: Application of D. newyorkensis NYU-BL-A4 in Prolonging the Lifespan of C. elegans

(1) Strain activation: D. newyorkensis NYU-BL-A4 cryopreserved at −80° C. was inoculated in modified MTGE broth medium (MTGE broth medium+0.05% cysteine hydrochloride), cultured for 48 h under anaerobic condition at 37° C., and then passaged with an inoculum of 2%-4% (v/v) in liquid for 2 times to fully activate the strain.

(2) Recovery, culture and passage of C. elegans

1 tube of nematodes in the dauer period (rest period) was taken from the refrigerator at −80° C. and placed in a 37° C. water bath for 1 min to be quickly melted, and then spread onto an NGM medium (nematode growth medium) containing uracil-deficient Escherichia coli OP50 and cultured in a biochemical incubator at 20° C. If a single nematode was passaged, one nematode was picked and placed on a new NGM medium by a self-made diamond gold wire (diameterization was performed for 3 min) burned with an alcohol lamp; if a large number of passages were required, the NGM medium for culturing nematodes was cut off by a surgical blade burned with alcohol lamp flame and adhered to a new medium.

(3) Synchronization of C. elegans

The well-growing adults were collected in a centrifuge tube, and 1 mL M9 buffer was added to wash off the excess E. coli OP50. 750 μL of the supernatant were retained in the centrifuge tube and 50 μL 1M KOH solution and 200 μL 5% NaClO solution were added. The mixture was shaken on a Vortex oscillator for about 3 min and tested under a stereomicroscope while shaking. If the nematode body was broken, it was washed with M9 buffer for 3-5 times as soon as possible and then centrifuged with a microcentrifuge to remove the excess solution and leave the eggs. The eggs were added with 500 μL of M9 buffer and placed on a shaker at 20° C. The eggs hatched into synchronized L1 larvae at 16 h.

(4) Detection of the lifespan of C. elegans

The synchronized nematodes were fed to the L4 stage on an NGM plate coated with E. coli OP50, and then moved to the NGM mediums respectively coated with 1.0×10⁸ CFU/mL of E. coli OP50 and 3.0×10⁸ CFU/mL of D. newyorkensis NYU-BL-A4. The NGM medium used in this experiment contained 50 mg/mL FUDR (Fluorouridine), and 20 nematodes were cultured in each petri dish in triplicate. The number of live nematodes was counted every 2 days in the lifespan experiment model and the medium was replaced with a new medium. At the end of the experiment, a lifespan curve of nematodes was drawn.

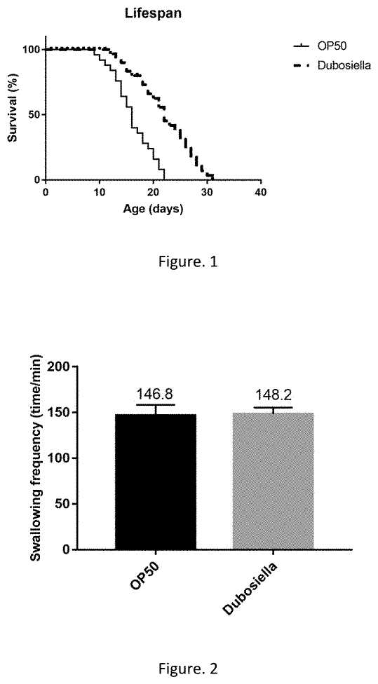

As shown in FIG. 1, the lifespan of the nematodes fed by D. newyorkensis NYU-BL-A4 is significantly longer than that of the control group. Upon calculation, the average lifespan of the control group is 16 days and the average lifespan of the experimental group is 22 days. It can be seen that D. newyorkensis NYU-BL-A4 has the effect of prolonging lifespan.

### Example 2: Effect of D. newyorkensis NYU-BL-A4 on the Vital Sign of Swallowing Frequency of C. elegans

The methods of recovery, culture and synchronization of nematodes were the same as those described in Example 1.

In order to verify whether the lifespan prolonging effect of D. newyorkensis is achieved by restricting the food intake of the nematode, when the swallowing rhythm of the nematode decreases, it will lead to insufficient food intake, which will greatly prolong the lifespan. Detection of the swallowing frequency of the nematodes: the synchronized nematodes cultured to L2 to L3 stages were taken and transferred to the NGM mediums respectively coated with 1.0×10⁸ CFU/mL of E. coli OP50 and 3.0×10⁸ CFU/mL of D. newyorkensis NYU-BL-A4. The nematodes were observed under a stereomicroscope and the number of swallows within 1 min was counted, in which each group was counted for 10 times. The statistical results were shown in FIG. 2. The average number of times in the control group is 146 times, and the average number of times in the experimental group is 148 times. Therefore, D. newyorkensis NYU-BL-A4 does not affect the normal vital sign of swallowing frequency of C. elegans, and does not prolong the lifespan by restricting food intake.

### Example 3: Detection of D. newyorkensis NYU-BL-A4 on the Safety to C. elegans

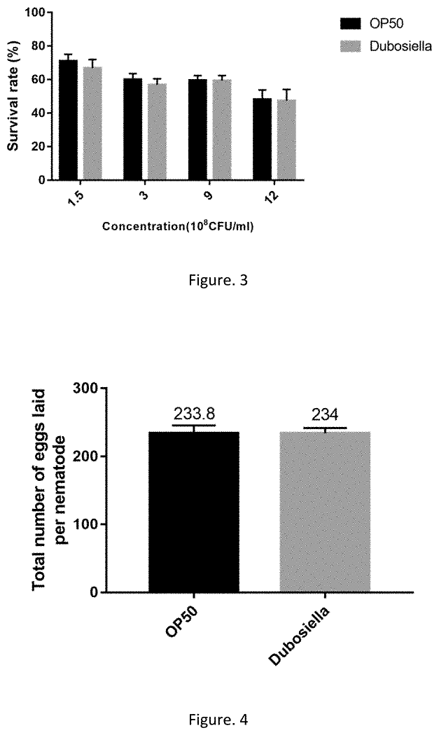

Detection of the toxicity to nematodes: E. coli OP50 and D. newyorkensis NYU-BL-A4 bacterial solutions were adjusted to different concentration (concentration was 1.5×10⁸ CFU/mL, 3×10⁸ CFU/mL, 9×10⁸ CFU/mL, and 12×10⁸ CFU/mL, respectively) with M9 buffer, the bacterial solutions with different concentration were spread on the NGM plate medium for synchronization, respectively. About 25 synchronized nematodes at L4 stage were placed on each plate. After 10 days, the number of nematode survivals was recorded and the survival rate was calculated. The results are shown in FIG. 3, which shows that the survival rates of nematodes fed with E. coli OP50 and D. newyorkensis under the same concentration conditions are kept at the same level. It can be considered that D. newyorkensis NYU-BL-A4 is safe for nematodes and does not affect their normal life activities.

### Example 4: D. newyorkensis NYU-BL-A4 does not Affect the Reproductive Ability of C. elegans

The synchronized eggs were cultured to the L4 stage at 20° C., and one nematode at L4 stage was picked and placed on an NGM culture plate coated with 3.0×10⁸ CFU/mL of D. newyorkensis or E. coli OP50 (one nematode per plate), and the spawning adults were transferred to a new culture plate every 24 hours until the nematode spawning was completely finished.

When the offspring nematodes were cultured to the L2-L3 stages, the number was counted. The number of offspring nematodes was the number of eggs laid by the mother nematodes. Finally, the sum of the number of eggs laid by the nematodes per day and the number of eggs laid during the spawning period was obtained. In each group of experiments, 3 nematodes were selected for simultaneous reproductive ability testing. This experiment was repeated three times independently.

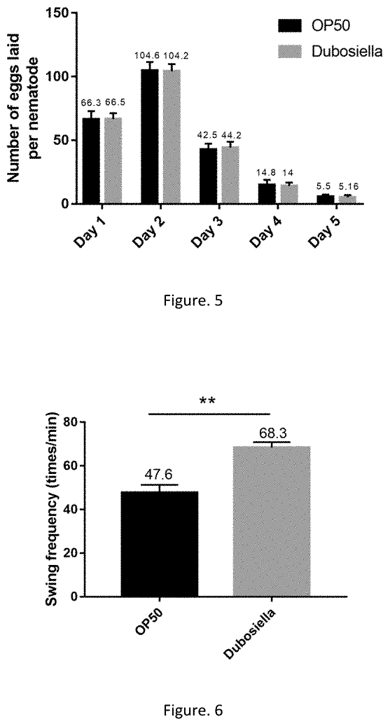

The results for nematode spawning experiment: the number of eggs laid by the nematodes is the reference basis for nematode reproductive capacity, and also the standard for evaluating nutrition and physical condition in nematodes. Studies have shown that reproductive ability is closely related to longevity. The screening of anti-aging drugs or strains should be based on the prerequisite of not damaging the reproductive ability of the body. For this reason, the effect of D. newyorkensis on the egg production ability of nematodes was evaluated. It can be seen from the results in FIGS. 4 and 5 that the peak of nematode reproduction after feeding D. newyorkensis is mainly concentrated in the first 3 days, and its egg production reaches its maximum on the second day. D. newyorkensis does not change the spawning period of the nematodes, and has no significant effect on the number of eggs laid by the nematodes per day and the total number of eggs laid. Therefore, it can be concluded that D. newyorkensis has no effect on the reproductive ability of nematodes.

### Example 5: D. newyorkensis NYU-BL-A4 Improves the Dynamic Swing Frequency of C. elegans

Experimental method: Synchronized nematodes at L4 stage were cultured on NGM culture plates respectively coated with 3.0×10⁸ CFU/mL of D. newyorkensis or E. coli OP50 for 10 days and then a 12-well plate was taken. 2 mL M9 buffer was added to each well, and 15 nematodes were placed in each well. The body swing frequency was observed within five min after placing, that is, the number of times that the nematode body bent and swung within one min (the body bent from one direction to another, and then restored to the original direction, which was recorded as one swing). The experiment was repeated 3 times independently.

With the aging of the nematodes, the muscles of the nematodes are degraded and their activity is slowed down. In the later stages of life, the nematodes move slowly and are not sensitive to external stimuli. The dynamic swing frequency of C. elegans in M9 buffer can explain the degree of aging to a certain extent. The results are shown in FIG. 6, compared with the blank group, the body swing frequency of nematodes fed with D. newyorkensis is significantly increased, and the swing frequency is increased by 43.49%. It shows that D. newyorkensis improves the activity of nematodes, has a positive effect on muscle function, and reduces the effect of aging on the muscles of nematodes to a certain extent.

Although the present disclosure has been disclosed as above in preferred examples, it is not intended to limit the present disclosure. Anyone familiar with this technology can make various changes and modifications without departing from the spirit and scope of the present disclosure. Therefore, the protection scope of the present disclosure should be defined by the claims.

What is claimed is:

1. A method of preparing products for prolonging lifespan and/or anti-aging in animals, wherein the products comprise D. newyorkensis NYU-BL-A4, and the D. newyorkensis NYU-BL-A4 was preserved in Leibniz Institute DSMZ-German Collection of Microorganisms and Cell Cultures, Inhoffenstraße 7B 38124 Braunschweig GERMANY on Jul. 8, 2016 and the preservation number is No. DSM 103457.

2. The method according to claim 1, wherein the products comprise food, health-care products, medicines and/or pharmaceutical compositions, feeds or additives thereof.

3. The method according to claim 2, wherein the food, health-care products, feeds or additives thereof are used to improve the immunity and strengthen the basal metabolic capacity in the body.

4. The method according to claim 3, wherein forms of the food, health-care products, feeds or additives thereof comprise tablets, granules, capsules, powders, liquids, jellies, and sticks.

5. The method according to claim 4, wherein the food, health-care products, feeds or additives thereof also contain common ingredients in the art, which are appropriately selected by those skilled in the art according to the dosage form or purpose of use, and can be used together with other raw materials.

6. The method according to claim 5, wherein prolonging lifespan is to prolong the maximum length of the lifespan of the body or delay the time to death.

7. The method according to claim 6, wherein the animal generally refers to organisms belonging to the animal kingdom in biological classification, and comprises mammals.

8. The method according to claim 7, wherein the content of D. newyorkensis in the product is not less than 1.0×10⁸ CFU/mL or 1.0×10⁸ CFU/mg.

9. A method of preparing products for improving the muscle capacity of animals, wherein the products comprise D. newyorkensis NYU-BL-A4, and the D. newyorkensis NYU-BL-A4 was preserved in Leibniz Institute DSMZ-German Collection of Microorganisms and Cell Cultures, Inhoffenstraße 7B 38124 Braunschweig GERMANY on Jul. 8, 2016 and the preservation number is No. DSM 103457.

10. The method according to claim 9, wherein the products comprise food, health-care products, medicines and/or pharmaceutical compositions, feeds or additives thereof.

11. The method according to claim 10, wherein forms of the food, health-care products, medicines and/or pharmaceutical compositions, feeds or additives thereof comprise tablets, granules, capsules, powders, liquids, jellies, and sticks.

12. The method according to claim 9, wherein the food, health-care products, feeds or additives thereof also contain common ingredients in the art, which are appropriately selected by those skilled in the art according to the dosage form or purpose of use, and can be used together with other raw materials.
